(19) United States
(12) Patent Application Publication
(10) Pub. No.: US 2026/0008765 A1
(43) Pub. Date: Jan. 8, 2026

(54) METHOD FOR PREPARING AND SEPARATING ALKYLENE CARBONATE USING CARBON DIOXIDE IN AIR

(71) Applicant: KOREA INSTITUTE OF SCIENCE AND TECHNOLOGY, Seoul (KR)

(72) Inventors: Hyun Joo LEE, Seoul (KR); Ung LEE, Seoul (KR); Chun-Jae YOO, Seoul (KR); Dahye WON, Seoul (KR); Kyeongsu KIM, Seoul (KR); Jiyun KIM, Seoul (KR)

(21) Appl. No.: 18/932,552

(22) Filed: Oct. 30, 2024

(30) Foreign Application Priority Data
Jul. 5, 2024 (KR) 10-2024-0088737

(51) Int. Cl. C07D 317/36; B01D 53/14; B01D 53/62; B01D 53/78; B01J 31/02; C07D 317/38; C07D 317/46

(52) U.S. Cl. CPC C07D 317/36; B01D 53/1475; B01D 53/1493; B01D 53/62; B01D 53/78; B01J 31/0279; B01J 31/0298; C07D 317/38; C07D 317/46; B01D 2252/2041; B01D 2252/20421; B01D 2257/504; B01D 2258/06

(57) ABSTRACT

A method for preparing and separating alkylene carbonate using carbon dioxide in air is disclosed herein. The method comprises the steps of: injecting air containing carbon dioxide into an amine solution containing a diamine compound to obtain a solution in which carbon dioxide is captured; adding alkylene oxide, a catalyst, and a solvent to the solution in which carbon dioxide is captured, and reacting to obtain a solution in which alkylene carbonate is produced; and separating alkylene carbonate from the solution in which alkylene carbonate is produced.

### CROSS REFERENCE TO RELATED APPLICATION

The present application claims the priority of Korean Patent Application No. 10-2024-0088737, filed on Jul. 5, 2024, the entire contents of which are incorporated herein for all purposes by this reference.

### BACKGROUND OF THE INVENTION

### Field of the Invention

The present disclosure discloses a method for preparing and separating alkylene carbonate using carbon dioxide in air.

### DESCRIPTION OF GOVERNMENT-SPONSORED RESEARCH

This research was conducted at the Korea Institute of Science and Technology under the management of the National Research Foundation of Korea which is affiliated with the Ministry of Science and ICT. The research business name is DACU Source Technology Development (R&D), and the research project name is Development of Source Technology for Simultaneous Capture and Conversion of Carbon Dioxide in Air (Unique Project Identification Number: 1711197843, Project Identification Number: 00259920).

### DESCRIPTION OF THE RELATED ART

Organic carbonate including ethylene carbonate is a substance which is widely used in the chemical and material industries, such as an electrolyte solvent for a secondary battery. The global market size of ethylene carbonate was recorded as approximately $450 million in 2021, and is showing a steep upward trend every year. In particular, it is expected to form a scale of $1.35 billion in 2030, which is increased to approximately three times compared to 2021. As the secondary battery industry grows, the demand for ethylene carbonate is increasing significantly, and hundreds of thousands of tons are produced annually in Korea. However, the technology for producing alkylene carbonate using carbon dioxide in air as a raw material, which can greatly contribute to alleviating global warming, is still insufficient.

In the past, alkylene carbonate had been prepared using carbon dioxide of a high concentration. The carbon dioxide of a high concentration has been obtained by separating carbon dioxide from a flue gas emitted from a power plant. The flue gas usually contains 10% to 15% of carbon dioxide, and in order to separate and concentrate it at a high concentration, an absorption-stripping process must be performed using a carbon dioxide absorbent such as an amine solution. 80% out of the energy which is consumed in this entire carbon dioxide capture system occurs in the stripping process. Therefore, it is necessary to develop a technology for a method capable of preparing alkylene carbonate directly using a solution in which carbon dioxide is captured without stripping and a method capable of preparing alkylene carbonate with less energy.

### SUMMARY OF THE INVENTION

In an aspect, the present disclosure aims to provide a method for preparing and separating alkylene carbonate from carbon dioxide in air.

In an aspect, the present disclosure provides a method for preparing and separating alkylene carbonate using carbon dioxide in air, the method comprising the steps of: injecting air containing carbon dioxide into a solution containing a carbon dioxide capturing agent to obtain a solution in which carbon dioxide is captured; adding alkylene oxide, a catalyst for producing alkylene carbonate, and a solvent to the solution in which carbon dioxide is captured, and reacting to obtain a solution in which alkylene carbonate is produced; and separating alkylene carbonate from the solution in which alkylene carbonate is produced.

In an exemplary embodiment, the carbon dioxide capturing agent may be a diamine compound represented by the following chemical formula 1:

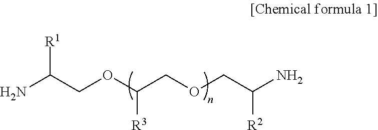

wherein n is any one of integers selected from 1 to 3,
R¹ and R² are the same and are a hexylamine group, a methyl group, an ethyl group, a propyl group or a butyl group, and
R³ is a methyl group.

In an exemplary embodiment, the solution containing the carbon dioxide capturing agent may be a solution in which the carbon dioxide capturing agent is dissolved in at least one solvent selected from the group consisting of monoglyme, diglyme, triglyme, tetraglyme, propylene carbonate, 1,4-dioxane, THF, and 2-methyltetrahydrofuran.

In an exemplary embodiment, the solution containing the carbon dioxide capturing agent may have a boiling point of 160 to 300° C.

In an exemplary embodiment, the catalyst for producing alkylene carbonate may be a quaternary ammonium halide.

In an exemplary embodiment, the quaternary ammonium halide may include at least one selected from the group consisting of cetyltrimethylammonium bromide (CTAB), tetraethylammonium bromide (TEAB), tetrapropylammonium bromide (TPAB), tetrabutylammonium bromide (TBAB), tetrabutylammonium chloride (TBACl), and tetra-n-butylammonium iodide (TBAI).

In an exemplary embodiment, the solvent may be at least one selected from the group consisting of monoglyme, diglyme, triglyme, tetraglyme, propylene carbonate, 1,4-dioxane, THF, and 2-methyltetrahydrofuran.

In an exemplary embodiment, the step of obtaining the solution in which alkylene carbonate is produced may include adding the alkylene oxide, the catalyst for producing alkylene carbonate, and the solvent to the solution in which carbon dioxide is captured, and reacting at 80 to 160° C. under an inert gas atmosphere.

In an exemplary embodiment, the inert gas may include at least one selected from the group consisting of hydrogen, nitrogen, helium, argon, and neon.

In an exemplary embodiment, the inert gas atmosphere is formed by injecting the inert gas to adjust a reactor pressure to 6 to 12 bar.

In an exemplary embodiment, the reaction may be carried out for 2 to 12 hours.

In an exemplary embodiment, the step of separating alkylene carbonate may be to inject air containing carbon dioxide into the solution in which the alkylene carbonate is produced to obtain a separated solvent layer and gel layer, wherein the solvent layer includes the solvent and the alkylene carbonate, and the gel layer includes the carbon dioxide capturing agent, the carbon dioxide, and the catalyst.

In an exemplary embodiment, the solvent layer and the gel layer may be obtained by decantation.

In an exemplary embodiment, the method may further comprise the step of adding the alkylene oxide and the solvent to the obtained gel layer and reacting to obtain a solution in which alkylene carbonate is produced.

In an exemplary embodiment, the alkylene oxide may be represented by the following chemical formula 2, and the alkylene carbonate may be represented by the following chemical formula 3:

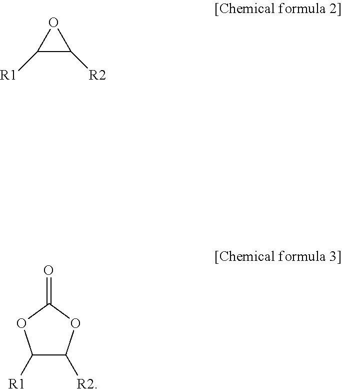

In the above chemical formulas 2 and 3, R¹ and R² are each independently hydrogen, a substituted or unsubstituted alkyl group having 1 to 6 carbon atoms, a substituted or unsubstituted haloalkyl group having 1 to 6 carbon atoms, a substituted or unsubstituted hydroxyalkyl group having 1 to 6 carbon atoms, a substituted or unsubstituted aryl group having 6 to 12 carbon atoms, or a substituted or unsubstituted arylalkyl group having 7 to 13 carbon atoms.

In an aspect, the technology described in the present disclosure has an effect of providing a method for preparing and separating alkylene carbonate from carbon dioxide in air. The method prepares alkylene carbonate directly using a solution in which carbon dioxide is captured without stripping, thereby having an effect of lowering a concentration of carbon dioxide in an atmosphere, which causes a problem in global warming, by using carbon dioxide in air rather than a flue gas.

In other aspect, the technology described in the present disclosure has an effect of providing a method for preparing alkylene carbonate using carbon dioxide in air, and then easily separating and purifying the alkylene carbonate by reinjecting the carbon dioxide in air to cause layer separation.

In another aspect, the technology described in the present disclosure has an effect of enabling continuous preparation of alkylene carbonate by reusing the carbon dioxide capturing agent and the catalyst which were obtained by the layer separation, thereby making it possible to perform a recycling reaction.

### BRIEF DESCRIPTION OF THE DRAWINGS

FIG. 1 shows a structural formula of an aliphatic quaternary ammonium bromide catalyst used in an embodiment.

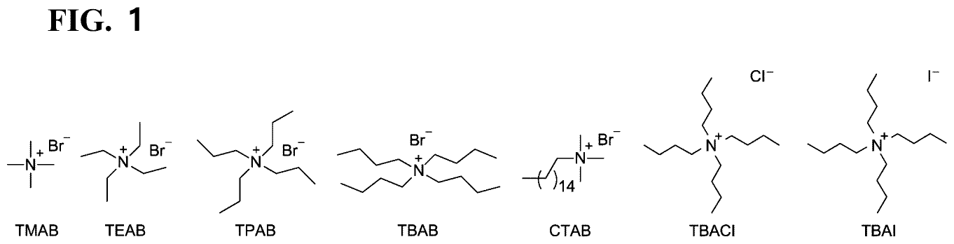

FIG. 2 shows a flow chart of a method for preparing and separating alkylene carbonate according to an embodiment. If carbon dioxide is reinjected into a solution in which alkylene carbonate is produced after the reaction is completed, phase separation occurs into a solvent layer and an amine gel layer in which carbon dioxide is captured, wherein the solvent layer and the amine gel layer can be separated by decantation. In addition, alkylene oxide and a solvent can be added to the separated amine gel layer to produce alkylene carbonate again, thereby making it possible to perform a recycling reaction.

FIG. 3 shows step-by-step photographs of a method for preparing and separating alkylene carbonate according to an embodiment.

FIG. 4 shows ¹H NMR spectra for reaction products of the compound of chemical formula 1-1 with propylene oxide (PO) according to a comparative embodiment. The graph shows the spectrum for tetrabutylammonium bromide (TBAB), the compound of chemical formula 1-1, a reaction product of the compound of chemical formula 1-1 without CO₂ capture with propylene oxide, and a reaction product of the compound of chemical formula 1-1 after CO₂ capture with propylene oxide, sequentially from bottom to top (PC (▾), TBAB (●)).

FIG. 5 shows ¹H NMR spectra for reaction products of 2-(tert-Butylamino) ethanol (t-BAE) with propylene oxide (PO) according to a comparative embodiment. The graph shows the spectrum for tetrabutylammonium bromide (TBAB), t-BAE, and a reaction product of t-BAE without CO₂ capture with propylene oxide, sequentially from bottom to top (TBAB (●), t-BAE (*), t-BAE+PO reaction product (▾)).

### DETAILED DESCRIPTION OF THE INVENTION

Hereinafter, the present disclosure will be described in detail.

In an aspect, the present disclosure provides a method for preparing and separating alkylene carbonate using carbon dioxide in air, the method comprising the steps of: injecting air containing carbon dioxide into a solution containing a carbon dioxide capturing agent to obtain a solution in which carbon dioxide is captured; adding alkylene oxide, a catalyst for producing alkylene carbonate, and a solvent to the solution in which carbon dioxide is captured, and reacting to obtain a solution in which alkylene carbonate is produced; and separating alkylene carbonate from the solution in which alkylene carbonate is produced.

The present disclosure provides a method for directly synthesizing alkylene carbonate by adding alkylene oxide to a solution in which carbon dioxide is captured from air. As shown in the following reaction scheme 1, the carbon dioxide and the alkylene oxide may be reacted in the presence of a catalyst to synthesize alkylene carbonate.

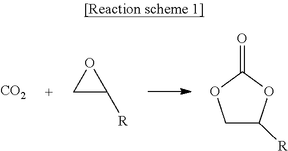

A process of capturing carbon dioxide according to the present disclosure must be performed under the absence of water. If water is present in the capture solution, it reacts with alkylene oxide to produce a glycol compound as shown in the following reaction scheme 2, and thus a system of capturing carbon dioxide without water is required. Therefore, a tertiary amine that can chemically capture carbon dioxide only in the presence of water is not suitable for the process of capturing carbon dioxide according to the present disclosure.

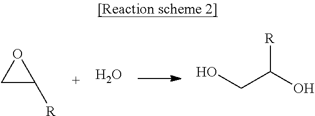

Further, since the representative CO₂ capturing amines such as monoethanolamine, diethanolamine, and aminomethylpropanol react with alkylene oxides to produce an alcohol compound as shown in the following reaction scheme 3, they are not suitable for the process of capturing carbon dioxide according to the present disclosure.

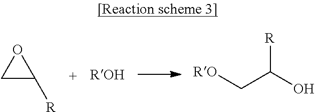

Furthermore, an amine that reacts with alkylene oxides is not suitable for the process of capturing carbon dioxide according to the present disclosure because a ring-opening reaction occurs by the amine as shown in the following reaction scheme 4.

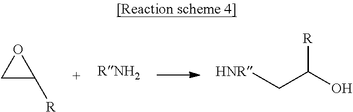

Therefore, in the process of capturing carbon dioxide according to the present disclosure, it may be preferable that the carbon dioxide capturing agent is a diamine compound containing an ether group.

In an exemplary embodiment, the carbon dioxide capturing agent may be a diamine compound represented by the following chemical formula 1:

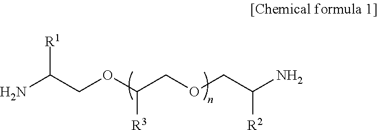

wherein n is any one of integers selected from 1 to 3,
R¹ and R² are the same and are a hexylamine group, a methyl group, an ethyl group, a propyl group or a butyl group, and
R³ is a methyl group.

In the method for preparing and separating alkylene carbonate using atmospheric carbon dioxide according to the present disclosure, the carbon dioxide capturing agent provides an effect capable of capturing not only carbon dioxide of a high concentration but also carbon dioxide having a low concentration of about 450 ppm in air. The carbon dioxide capturing agent can capture carbon dioxide of a low concentration and dissociate with carbon dioxide. Therefore, the carbon dioxide capturing agent can be separated from a product generated in a subsequent process, thereby providing an effect capable of preparing and separating alkylene carbonate from carbon dioxide in air.

In an exemplary embodiment, the carbon dioxide capturing agent may be a diamine compound represented by the following chemical formula 1-1, 1-2 or 1-3:

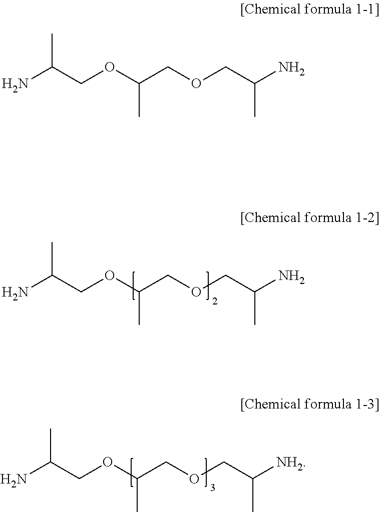

In an exemplary embodiment, the solution containing the carbon dioxide capturing agent may be a solution in which the carbon dioxide capturing agent is dissolved in at least one solvent selected from the group consisting of monoglyme, diglyme, triglyme, tetraglyme, propylene carbonate, 1,4-dioxane, THF, and 2-methyltetrahydrofuran.

In an exemplary embodiment, the solution containing the carbon dioxide capturing agent may have a boiling point of 160 to 300° C. Accordingly, the present disclosure has effects of preventing the possibility of loss along with air during the process of capturing carbon dioxide as the boiling point of the solution is lower than 160° C., and preventing a problem that a capturing rate of CO₂ becomes too slow due to a high viscosity of the solution when the boiling point of the solution exceeds 300° C.

In an exemplary embodiment, the catalyst for producing the alkylene carbonate may be a quaternary ammonium halide. Accordingly, when layer separation is formed after the reaction, the catalyst is present in an amine gel layer so that an alkylene oxide product contained in a solvent layer can be purely separated, which results in an effect capable of recycling of the amine gel layer containing the carbon dioxide capturing agent and the catalyst.

In an exemplary embodiment, the quaternary ammonium halide may be an aliphatic quaternary ammonium halide.

In an exemplary embodiment, the quaternary ammonium halide may be an aliphatic quaternary ammonium halide having 2 to 4 carbon atoms.

In an exemplary embodiment, the quaternary ammonium halide may be an aliphatic quaternary ammonium bromide, chloride, or iodide.

In an exemplary embodiment, the quaternary ammonium halide may include at least one selected from the group consisting of cetyltrimethylammonium bromide (CTAB), tetraethylammonium bromide (TEAB), tetrapropylammonium bromide (TPAB), tetrabutylammonium bromide (TBAB), tetrabutylammonium chloride (TBACl), and tetra-n-butylammonium iodide (TBAI).

In an exemplary embodiment, the quaternary ammonium halide may be tetraethylammonium bromide (TEAB).

In general, there was a problem that since the amine had a high boiling point and the produced alkylene carbonate also had a high boiling point (for example, ethylene carbonate: 243° C., propylene carbonate: 242° C.), it was difficult to separate them after the reaction. The method according to the present disclosure uses the solvent together with the catalyst for producing alkylene carbonate to facilitate the separation of alkylene carbonate.

In an exemplary embodiment, the solvent which is added to the solution in which carbon dioxide is captured together with the alkylene oxide and the catalyst for producing alkylene carbonate may be the same as or different from the solvent used in the solution containing the carbon dioxide capturing agent.

In an exemplary embodiment, the solvent which is added to the solution in which carbon dioxide is captured together with the alkylene oxide and the catalyst for producing alkylene carbonate may be at least one selected from the group consisting of monoglyme, diglyme, triglyme, tetraglyme, propylene carbonate, 1,4-dioxane, THE, and 2-methyltetrahydrofuran.

In an exemplary embodiment, the solvent which is added to the solution in which carbon dioxide is captured together with the alkylene oxide and the catalyst for producing alkylene carbonate may be diglyme (diethyleneglycoldimethylether).

In an exemplary embodiment, the step of obtaining a solution in which alkylene carbonate is produced may be to add the alkylene oxide, the catalyst for producing alkylene carbonate, and the solvent to the solution in which carbon dioxide is captured, and react at 80 to 160° C., 80 to 140° C., 80 to 120° C., or 80 to 100° C. under an inert gas atmosphere. The method for preparing and separating alkylene carbonate according to the present disclosure provides an effect of producing alkylene carbonate by adding the alkylene oxide, the catalyst, and the solvent to an amine solution into which carbon dioxide in air is dissolved, without using a process for concentrating carbon dioxide that consumes a large amount of energy, and then easily separating and purifying the alkylene carbonate through layer separation by injecting carbon dioxide in air again.

In an exemplary embodiment, the inert gas may include at least one selected from the group consisting of hydrogen, nitrogen, helium, argon, and neon.

In an exemplary embodiment, the inert gas atmosphere is formed by injecting the inert gas to adjust a reactor pressure to 6 to 12 bar.

In an exemplary embodiment, the reaction may be carried out for 2 to 12 hours, 2 to 10 hours, or 4 to 10 hours.

In an exemplary embodiment, the step of separating alkylene carbonate may be to inject air containing carbon dioxide into the solution in which the alkylene carbonate is produced to obtain a separated solvent layer and gel layer, wherein the solvent layer may include the solvent and the alkylene carbonate, and the gel layer may include the carbon dioxide capturing agent, the carbon dioxide, and the catalyst. The method for preparing and separating alkylene carbonate according to the present disclosure is to separates the alkylene carbonate through phase conversion and layer separation by injecting carbon dioxide in air again after producing the alkylene carbonate from carbon dioxide in air. The method provides an effect of producing the alkylene carbonate with a high efficiency using carbon dioxide in air rather than a flue gas, and then easily separating and purifying the alkylene carbonate through phase conversion and layer separation.

In an exemplary embodiment, the solvent layer and the gel layer may be obtained by decantation.

In an exemplary embodiment, the method may further comprise the step of adding the alkylene oxide and the solvent to the obtained gel layer and proceeding with a reaction to obtain a solution in which alkylene carbonate is produced. Thereafter, the carbon dioxide in the air may be reinjected as in the subsequent process described above to separate and purify the alkylene carbonate through layer separation. In the method according to the present disclosure, since the carbon dioxide capturing agent, which is a diamine compound containing an ether group, captures the carbon dioxide in air well, it can be separated from the reaction product for reuse thereof. The present disclosure has an effect capable of performing a recycling reaction by reusing the carbon dioxide capturing agent and the catalyst.

In an exemplary embodiment, the alkylene oxide may be represented by the following chemical formula 2, and the alkylene carbonate may be represented by the following chemical formula 3:

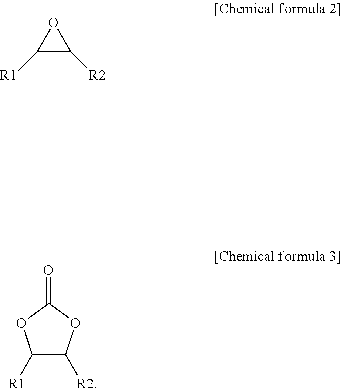

In the above chemical formulas 2 and 3, R¹ and R² are each independently hydrogen, a substituted or unsubstituted alkyl group having 1 to 6 carbon atoms, a substituted or unsubstituted haloalkyl group having 1 to 6 carbon atoms, a substituted or unsubstituted hydroxyalkyl group having 1 to 6 carbon atoms, a substituted or unsubstituted aryl group having 6 to 12 carbon atoms, or a substituted or unsubstituted arylalkyl group having 7 to 13 carbon atoms.

In an exemplary embodiment, R¹ and R² in the chemical formulas 2 and 3 may be connected to each other to form a ring or not to form the ring.

In an exemplary embodiment, the substitution may be a substitution with an ether group.

In an exemplary embodiment, the alkylene oxide may be ethylene oxide, propylene oxide, or butylene oxide.

In an exemplary embodiment, the alkylene carbonate may be ethylene carbonate, propylene carbonate, or butylene carbonate.

Hereinafter, the present disclosure will be described in more detail through Examples. These Examples are intended to only illustrate the present disclosure, and will be apparent to those skilled in the art that the scope of the present disclosure should not be construed as being limited by these Examples.

### Example 1

An amine solution in which carbon dioxide was captured was obtained by dissolving a diamine compound of the following chemical formula 1-1 (1.2 g, 6.3 mmol) as a carbon dioxide capturing agent in 15 mL of a diglyme solvent, and then treating air containing 430 ppm of carbon dioxide at 500 cc/min. An amount of carbon dioxide captured over time was shown in Table 1 below.

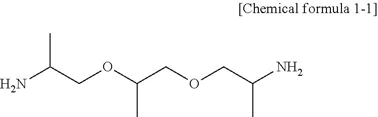

TABLE 1

| Time | Amount of CO₂ captured (CO₂ mol/amine mol) |
|---|---|
| 4 | 0.12 |
| 6 | 0.32 |
| 12 | 0.74 |
| 24 | 1.03 |

### Example 2

Propylene oxide (4.5 g, 77.5 mmol) as alkylene oxide, an aliphatic quaternary ammonium bromide catalyst (0.5 mmol) listed in Table 2 below, and 15 g of a diglyme solvent were added to the amine solution in which carbon dioxide was captured by treating them for 24 hours in Example 1 above, and were reacted under a nitrogen atmosphere of 10 bar at 80° C. for 4 hours to obtain a solution in which propylene carbonate (PC) was produced.

Thereafter, air containing carbon dioxide was injected again into the solution in which propylene carbonate was produced for 24 hours, and a solution separated into a solvent layer and a gel layer was decantated. Through the layer separation, the solvent layer containing the solvent and the propylene carbonate and the gel layer containing the carbon dioxide capturing agent, the carbon dioxide, and the catalyst were obtained to perform ¹H NMR analysis (400 MHz, Brucker).

As a result, it was found that there was a difference in an amount of propylene carbonate produced or results of layer separation depending on types of the used catalysts (see Table 2). TMAB or TEAB having a short length of an alkyl group showed no or low reactivity, and when CTAB, TPAB, or TBAB was used as the catalyst, the propylene carbonate was detected on the diglyme layer, confirming that the PC conversion reactivity was excellent.

$$Yeild ( \% ) of propylene carbonate ( P C ) = 100 \times Amount of P C produced ( mmol ) / Amount of {CO}_{2} captured ( mmol )$$

TABLE 2

| Catalyst | Yield of PC (%) |
|---|---|
| NH₄Br | 20 |
| TMAB | 0 |
| CTAB | 76 |
| TEAB | 17 |
| TPAB | 100 |
| TBAB | 100 |

### Example 3

Alkylene carbonate was prepared and separated from carbon dioxide in air in the same method as that of Example 2, except that a diamine compound of chemical formula 1-2 or 1-3 was used as a carbon dioxide capturing agent to obtain an amine solution in which carbon dioxide was captured, and tetrabutylammonium bromide (TBAB) was used as a catalyst. After air containing 430 ppm of carbon dioxide was treated at 500 cc/min for 24 hours, an amount of carbon dioxide captured and a yield of propylene carbonate as a result of the reaction were shown in Table 3 below.

TABLE 3

| | Amount of CO₂ captured (CO₂ | |
|---|---|---|
| Carbon dioxide capturing agent | mol/amine mol) | Yield of PC (%) |
| [Chemical formula 1-2] 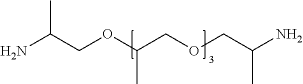 | 1.02 | 92 |
| [Chemical formula 1-3] 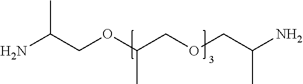 | 1.04 | 95 |

### Example 4

Alkylene carbonate was prepared and separated from carbon dioxide in air in the same method as that of Example 2, except that tetrabutylammonium bromide (TBAB), tetrabutylammonium chloride (TBACl), or tetra-n-butylammonium iodide (TBAI) was used as a catalyst and triglyme or tetraglyme was used as a solvent to obtain an amine solution in which carbon dioxide was captured and a solution in which propylene carbonate was produced. The results obtained by changing the catalyst and solvent as described above and reacting them were shown in Table 4 below.

TABLE 4

| Catalyst | Solvent | Yield of PC (%) |
|---|---|---|
| TBAB | Triglyme | 93 |
| TBACl | Tetraglyme | 86 |
| TBAI | Tetraglyme | 100 |

### Example 5

Alkylene carbonate was prepared and separated from carbon dioxide in air in the same method as that of Example 2, except that tetrabutylammonium bromide (TBAB) was used as a catalyst and the solvent given by Table 5 below was used as a solvent to obtain an amine solution in which carbon dioxide was captured and a solution in which propylene carbonate was produced. A yield of propylene carbonate as a result of the reaction was shown in Table 5 below.

TABLE 5

| Solvent | Solvent structure | Yield of PC (%) |
|---|---|---|
| Monoglyme | 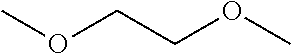 | 96 |
| Tetraglyme | 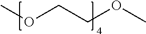 | 94 |
| Propylene carbonate | 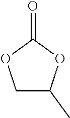 | 92 |
| 1,4-dioxane | 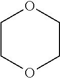 | 89 |
| THF | 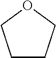 | 94 |
| 2-Methyltetrahydrofuran | 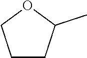 | 96 |

### Example 6

Alkylene carbonate was prepared and separated from carbon dioxide in air in the same method as that of Example 2, except that tetrabutylammonium bromide (TBAB) was used as a catalyst and the compound shown in Table 6 below was used as alkylene oxide to prepare alkylene carbonate. A reaction temperature and a reaction time were as shown in Table 6 below, and a yield of the alkylene carbonate produced as a result of the reaction was shown in Table 6 below.

$$Yeild ( \% ) of alkylene carbonate produced = 100 \times Amount of alkylene carbonate produced ( mmol ) / Amount of {CO}_{2} captured ( mmol )$$

TABLE 6

| | Reaction | | |
|---|---|---|---|
| | temperature and | Alkylene carbonate | Yield (%) of alkylene |
| Alkylene oxide | reaction time | produced | carbonate produced |
| 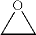 | 80° C., 4 h | 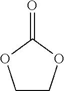 | 100 |
| 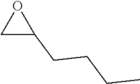 | 80° C., 4 h | 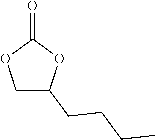 | 95 |
| 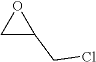 | 80° C., 9 h | 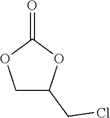 | 90 |
| 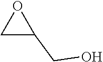 | 80° C., 9 h | 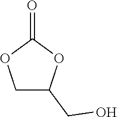 | 92 |
| 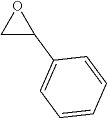 | 80° C., 9 h | 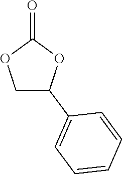 [SMILES: O=C1OCC(c2ccccc2)O1] | 99 |
| 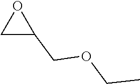 | 80° C., 9 h | 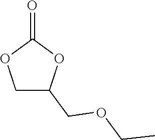 | 96 |
| 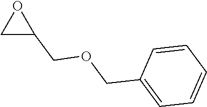 [SMILES: c1ccc(COCC2CO2)cc1] | 100° C., 9 h | 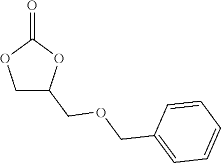 [SMILES: O=C1OCC(COCc2ccccc2)O1] | 91 |
| 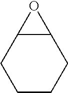 | 100° C., 9 h | 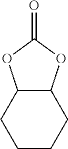 | 88 |

### Example 7

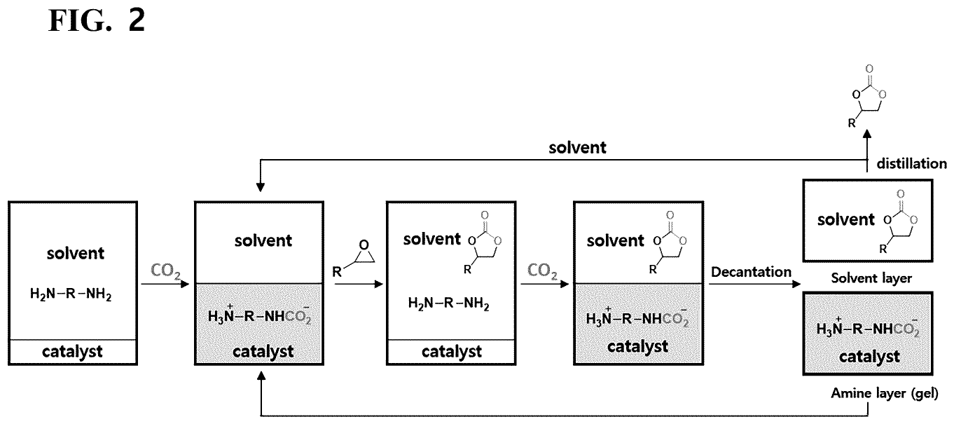

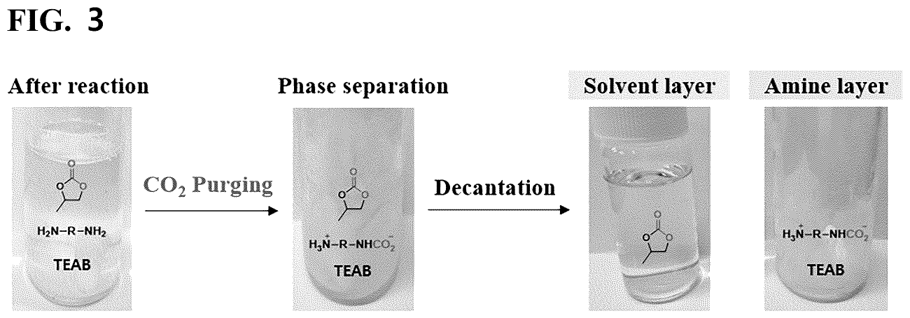

Alkylene carbonate was prepared and separated from carbon dioxide in air in the same method as that of Example 2, except that a conversion reaction into propylene carbonate was performed by using tetraethylammonium bromide (TEAB) as a catalyst and raising a reaction temperature to 120° C. when propylene oxide, the catalyst, and a solvent were added to perform the reaction (see FIGS. 2 and 3). A yield of propylene carbonate as a result of the reaction was shown in Table 7 below.

As a result of performing the conversion reaction into propylene carbonate by adding the propylene oxide, the catalyst, and the solvent and raising the reaction temperature, it was confirmed that the PC conversion reactivity was excellent even though TEAB having a short length of an alkyl group was used as the catalyst. Specifically, it was shown that when TEAB was used as the catalyst and the PC conversion reaction was performed at 80° C., the propylene carbonate was produced with a yield of 17% based on the captured CO₂, but when the reaction temperature was raised to 120° C., a yield of the propylene carbonate was increased by nearly four times.

After the conversion reaction into propylene carbonate, a white solid was observed in the solution where propylene carbonate was produced, which is expected to be TEAB. In addition, as a result of injecting CO₂ again into the solution in which propylene carbonate was produced, it was confirmed that layer separation occurred into two layers. The carbon dioxide capturing agent, propylene carbonate, and catalyst could be separated by injecting CO₂ into the reaction product existing as 1-phase to induce phase separation. The layer separation was classified into a solvent layer containing diglyme and propylene carbonate and an amine gel layer containing the carbon dioxide capturing agent and catalyst, and the result of NMR analysis showed that approximately 95% of a total amount of propylene carbonate produced was confirmed in the solvent layer. The catalyst TEAB was detected in the amine gel layer in which carbon dioxide was captured, and was not detected in the solvent layer, i.e., the diglyme layer. Therefore, propylene oxide and diglyme were added again to the separated amine gel layer to synthesize propylene carbonate, and it was confirmed that the recycling reaction was possible. As a result of reusing the carbon dioxide capturing agent and the catalyst as described above, it was confirmed that propylene carbonate could be produced in high yields in the second, third, and fourth reactions as shown in Table 7 below.

TABLE 7

| Number of reactions | Catalyst | Yield of PC (%) |
|---|---|---|
| 1st | TEAB | 69 |
| 2nd | TEAB | 75 |
| 3rd | TEAB | 72 |
| 4th | TEAB | 72 |

### Comparative Example 1

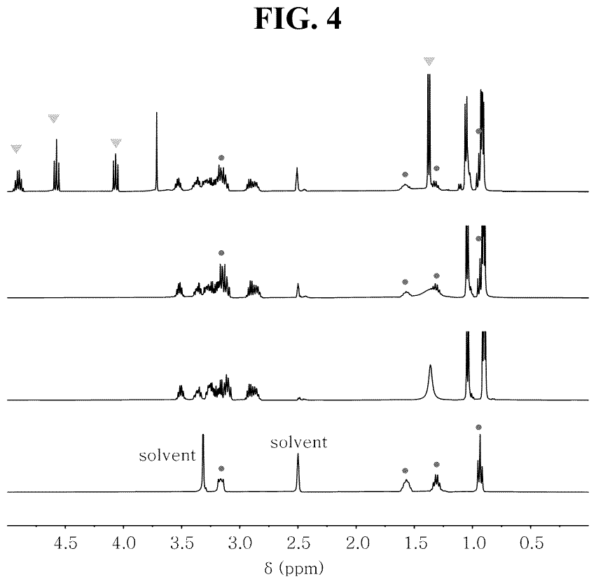

As a result of measuring 1H-NMR after reacting the compound of chemical formula 1-1 with propylene oxide in the presence of the catalyst TBAB at 80° C. for 4 hours, it was confirmed that only the compound of chemical formula 1-1 and the catalyst TBAB were detected as shown in the second spectrum from the top of FIG. 4, which shows that propylene carbonate was not produced. On the other hand, as a result of collecting CO₂ by using the compound of chemical formula 1-1 and then reacting them under the same condition, it was confirmed that propylene carbonate (PC) was produced as shown in the spectrum at the top of FIG. 4.

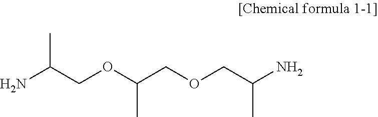

### Comparative Example 2

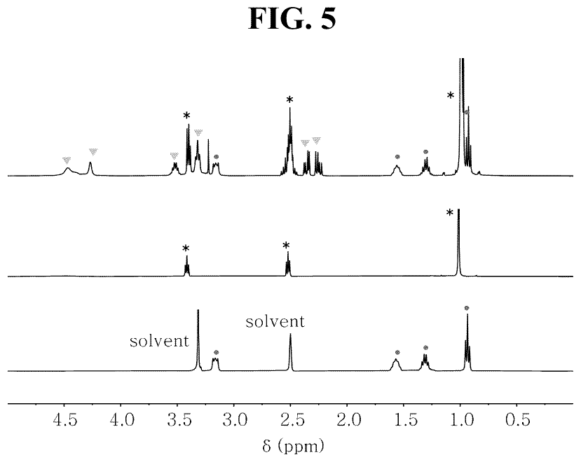

As a result of measuring 1H-NMR after reacting 2-(tert-Butylamino) ethanol (t-BAE) containing an alcohol group other than the compound of chemical formula 1-1 as a carbon dioxide capturing agent with propylene oxide (PO) in the presence of the catalyst TBAB at 80° C. for 4 hours, it was confirmed that the alcohol group of t-BAE reacted with PO to produce various substances (see FIG. 5). Accordingly, it could be seen that the carbon dioxide capturing agent capable of reacting with alkylene oxide was not suitable in the method for preparing and separating alkylene carbonate according to the present disclosure.

While the specific portions of the present disclosure have been described in detail above, it will be apparent to those skilled in the art that such specific descriptions are merely preferred embodiments and that the scope of the present disclosure is not limited thereby. Accordingly, the substantial scope of the present disclosure will be defined by the appended claims and their equivalents.

What is claimed is:

1. A method for preparing and separating alkylene carbonate using carbon dioxide in air, the method comprising the steps of:
injecting air comprising carbon dioxide into a solution comprising a carbon dioxide capturing agent to obtain a solution in which carbon dioxide is captured;
adding alkylene oxide, a catalyst for producing alkylene carbonate, and a solvent to the solution in which carbon dioxide is captured, and reacting to obtain a solution in which alkylene carbonate is produced; and
separating alkylene carbonate from the solution in which alkylene carbonate is produced.

2. The method for preparing and separating alkylene carbonate using carbon dioxide in air according to claim 1,
wherein the carbon dioxide capturing agent is a diamine compound represented by the following chemical formula 1: 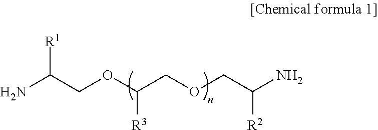
wherein n is any one of integers selected from 1 to 3,
R¹ and R² are the same and are a hexylamine group, a methyl group, an ethyl group, a propyl group, or a butyl group, and
R³ is a methyl group.

3. The method for preparing and separating alkylene carbonate using carbon dioxide in air according to claim 1,
wherein the solution comprising the carbon dioxide capturing agent is a solution in which the carbon dioxide capturing agent is dissolved in at least one solvent selected from the group consisting of monoglyme, diglyme, triglyme, tetraglyme, propylene carbonate, 1,4-dioxane, THF, and 2-methyltetrahydrofuran.

4. The method for preparing and separating alkylene carbonate using carbon dioxide in air according to claim 1,
wherein the solution comprising the carbon dioxide capturing agent has a boiling point of 160 to 300° C.

5. The method for preparing and separating alkylene carbonate using carbon dioxide in air according to claim 1,
wherein the catalyst for producing alkylene carbonate is a quaternary ammonium halide.

6. The method for preparing and separating alkylene carbonate using carbon dioxide in air according to claim 5,
wherein the quaternary ammonium halide comprises at least one selected from the group consisting of cetyltrimethylammonium bromide (CTAB), tetraethylammonium bromide (TEAB), tetrapropylammonium bromide (TPAB), tetrabutylammonium bromide (TBAB), tetrabutylammonium chloride (TBACl), and tetra-n-butylammonium iodide (TBAI).

7. The method for preparing and separating alkylene carbonate using carbon dioxide in air according to claim 1,
wherein the solvent is at least one selected from the group consisting of monoglyme, diglyme, triglyme, tetraglyme, propylene carbonate, 1,4-dioxane, THF, and 2-methyltetrahydrofuran.

8. The method for preparing and separating alkylene carbonate using carbon dioxide in air according to claim 1,
wherein the step of obtaining the solution in which alkylene carbonate is produced is adding the alkylene oxide, the catalyst for producing alkylene carbonate, and the solvent to the solution in which carbon dioxide is captured, and reacting at 80 to 160° C. under an inert gas atmosphere.

9. The method for preparing and separating alkylene carbonate using carbon dioxide in air according to claim 8,
wherein the inert gas comprises at least one selected from the group consisting of hydrogen, nitrogen, helium, argon, and neon.

10. The method for preparing and separating alkylene carbonate using carbon dioxide in air according to claim 8,
wherein the inert gas atmosphere is formed by injecting the inert gas to adjust a reactor pressure to 6 to 12 bar.

11. The method for preparing and separating alkylene carbonate using carbon dioxide in air according to claim 8,
wherein the reaction is carried out for 2 to 12 hours.

12. The method for preparing and separating alkylene carbonate using carbon dioxide in air according to claim 1,
wherein the step of separating alkylene carbonate is to inject air comprising carbon dioxide into the solution in which the alkylene carbonate is produced to obtain a separated solvent layer and gel layer,
wherein the solvent layer comprises the solvent and the alkylene carbonate, and the gel layer comprises the carbon dioxide capturing agent, the carbon dioxide, and the catalyst.

13. The method for preparing and separating alkylene carbonate using carbon dioxide in air according to claim 12,
wherein the solvent layer and the gel layer are obtained by decantation.

14. The method for preparing and separating alkylene carbonate using carbon dioxide in air according to claim 12,
further comprising the step of adding the alkylene oxide and the solvent to the obtained gel layer and reacting to obtain a solution in which alkylene carbonate is produced.

15. The method for preparing and separating alkylene carbonate using carbon dioxide in air according to claim 1,
wherein the alkylene oxide is represented by the following chemical formula 2, and the alkylene carbonate is represented by the following chemical formula 3: 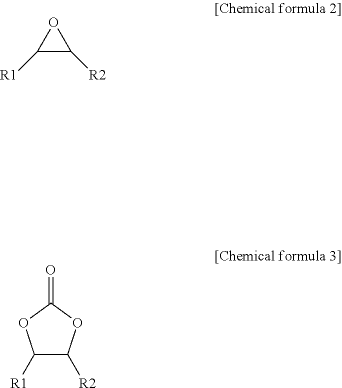
wherein R1 and R2 are each independently hydrogen, a substituted or unsubstituted alkyl group having 1 to 6 carbon atoms, a substituted or unsubstituted haloalkyl group having 1 to 6 carbon atoms, a substituted or unsubstituted hydroxyalkyl group having 1 to 6 carbon atoms, a substituted or unsubstituted aryl group having 6 to 12 carbon atoms, or a substituted or unsubstituted arylalkyl group having 7 to 13 carbon atoms.
